(19) United States
(12) Patent Application Publication
(10) Pub. No.: US 2007/0219441 A1
(43) Pub. Date: Sep. 20, 2007

(54) Catheter with integral biosensor

(76) Inventors: Patrick Carlin, Dana Point, CA (US); Michael J. Higgins, Huntington Beach, CA (US); Kenneth M. Curry, Oceanside, CA (US); Todd Fjield, Laguna Hills, CA (US); Harold A. Heitzmann, Irvine, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/710,834

(22) Filed: Feb. 26, 2007

Related U.S. Application Data
(60) Provisional application No. 60/777,030, filed on Feb. 27, 2006.

(51) Int. Cl. A61B 5/00; A61B 5/05; A61B 5/04

(52) U.S. Cl. 600/365; 600/347; 600/381

(57) ABSTRACT

A single or multilumen intravenous catheter that may include an integral biosensor having an active portion exposed through a sensing port formed in a distal portion of an outer wall of the catheter. The biosensor may be formed on a flex circuit mounted to a support member or probe that displaces the active portion from an inner wall of the catheter for protection from friction during installation through a lumen. The support member or probe may position the biosensor concentrically within the lumen or against an inner diameter of the outer wall. The biosensor may be sealed about the sensing port to prevent passage of fluid therethrough, or a proximal end of the sensing port may remain open to allow flushing of the biosensor with saline infused through the lumen.

Claim of Priority under 35 U.S.C. §119

The present Application for Patent claims priority to Provisional Application No. 60/777,030 filed Feb. 27, 2006, and assigned to the assignee hereof and hereby expressly incorporated by reference herein.

### FIELD OF THE INVENTION

The invention relates generally to catheters used in medical applications. More specifically, the invention relates to a multilumen central venous catheter (CVC) having an integral biosensor for detecting a physiological parameter.

### BACKGROUND

In medical applications, patients in intensive care units (ICUs) or other emergency situations are often fitted with invasive appliances such as catheters so that vital fluids or medicine may be administered intravenously. A physician determining a fluid dosage to be provided to a patient intravenously may need to know symptoms as quickly as possible that can only be determined through blood tests. Just how quickly the information is needed depends on the gravity of the situation. In some cases, the speed with which a physiological parameter can be determined may be the difference between life and death. In those situations, the practice of drawing a blood sample and sending it off for laboratory analysis may be entirely too slow.

A more timely method for measuring blood chemistry to ascertain a physiological parameter of interest may eventually be perfected. One promising area in this field is amperometry, or intravenous amperometric sensing, in which the concentration of a material present in a patient's bloodstream may be determined by locating, within the circulatory system, an enzyme electrode that produces an electrical current proportional to the material concentration. If successfully engineered, this type of sensor, or biosensor, could be monitored continuously over many hours, or perhaps even days, using analytical electronics coupled to the biosensor through a conductive interface.

Among many problems impeding the development of a practical intravenous amperometric biosensor is the spatial design constraint posed by the circulatory system. The biosensor needs to be small enough to be suspended within a blood vessel, and still have sufficient mechanical integrity to withstand the rigors of installation. In addition, an attending physician needs to be able to quickly position the biosensor in a location that will provide accurate measurements.

One approach to solving the positioning problem has been proposed in U.S. Patent Application Publication 2004/0064086, which is directed to a multilumen catheter fitted with a sensing element. This publication, however, provides little or no guidance regarding how to install the sensing element within the catheter.

Installing a biosensor within a catheter raises a number of other problems. Any shielding system employed to protect the biosensor from damage during installation may still expose the biosensor to a continuous flow of venous blood when in use. The system may also discourage blood from clotting around the exposed portion of the biosensor, and allows for a reliable electrical connection to external instrumentation to be maintained. In short, a reliable system for in situ positioning of an intravenous biosensor has yet to be developed.

### SUMMARY

The invention discloses a single lumen or multilumen intravenous catheter assembly that includes an integral biosensor. The biosensor may be an amperometric sensor formed on a flex circuit and having an active portion containing an enzyme electrode that reacts with a substance in blood, such as glucose, to measure a physiological parameter such as glucose concentration. The biosensor may be positioned on the insertion or distal end of the catheter within or adjacent to a lumen for exposure to blood when the catheter is installed in a blood vessel. Electrical wires secured to the flex circuit may energize the electrode and may carry signals indicative of the physiological parameter to an electrical connector disposed on the proximal end of the catheter. One or more infusion ports also located on the proximal end of the catheter may be provided to inject infusate through another lumen into a patient.

In one embodiment, the catheter may include an elongated tube that forms the insertion portion of the assembly. The biosensor may be exposed to blood through a sensing port perforating an outer wall of the catheter tube between its proximal and distal ends. A lumen may extend through the tube and connect to the sensing port. The biosensor may be mounted to a support member or probe that displaces the active portion from an inner wall of the catheter for protection from friction during installation of the biosensor through the lumen. The support member or probe may position the biosensor concentrically within the lumen or against an inner diameter of the outer wall, so that the active portion is protectively displaced from an inner wall of the catheter. The biosensor may be sealed about the sensing port to prevent passage of fluid therethrough, or a proximal end of the sensing port may remain open to allow flushing of the biosensor with saline infused through the lumen. Alternatively, the biosensor may be mounted in a recessed area formed in the outer wall. The sensing port or recessed area may be placed proximally to fluid ejection ports to prevent infusate from affecting intravenous biosensor measurements.

### BRIEF DESCRIPTION OF THE DRAWINGS

The features, objects, and advantages of the invention will become more apparent from the detailed description set forth below when taken in conjunction with the drawings, wherein:

FIG. 1 is a side view of a multilumen catheter assembly according to an embodiment of the invention.

FIG. 2 is a magnified detail of the distal end of the multilumen catheter of FIG. 1 according to an embodiment of the invention.

FIG. 3 is a magnified transparent side view of an intermediate portion of the distal end of the catheter of FIG. 1 in which a biosensor is centrally oriented within a lumen and exposed through an opening in the outer catheter wall according to an embodiment of the invention.

FIG. 4 is a transparent bottom view of the intermediate portion of FIG. 3 according to an embodiment of the invention.

FIG. 5 is a magnified cross sectional view of the catheter of FIG. 3 according to an embodiment of the invention.

FIG. 6 is a magnified transparent side view of an intermediate portion of the distal end of the catheter of FIG. 1 in which a biosensor is mounted to an inner wall of the catheter and exposed through an opening in the outer catheter wall according to an embodiment of the invention.

FIG. 7 is a transparent bottom view of the intermediate portion of FIG. 6 according to an embodiment of the invention.

FIG. 8 is a magnified cross sectional view of the catheter of FIG. 6 according to an embodiment of the invention.

FIG. 9 is a magnified transparent side view of an intermediate portion of the distal end of the catheter of FIG. 1 in which a biosensor is centrally oriented within a lumen open at the proximal side of the biosensor to allow for flushing of the biosensor according to an embodiment of the invention.

FIG. 10 is a transparent bottom view of the intermediate portion of FIG. 9 according to an embodiment of the invention.

FIG. 11 is a magnified cross sectional view of the catheter of FIG. 9 according to an embodiment of the invention.

FIG. 12 is a magnified transparent side view of an intermediate portion of the distal end of the catheter of FIG. 1 in which a biosensor is mounted to an outer wall of the catheter according to an embodiment of the invention.

FIG. 13 is a transparent bottom view of the intermediate portion of FIG. 12 according to an embodiment of the invention.

FIG. 14 is a magnified cross sectional view of the catheter of FIG. 12 according to an embodiment of the invention.

FIG. 15 is a magnified transparent side view of an intermediate portion of the distal end of the catheter of FIG. 1 in which a biosensor is integrated into a probe inserted through a lumen to position the biosensor coincident with an opening in the outer catheter wall according to an embodiment of the invention.

FIG. 16 is a transparent bottom view of the intermediate portion of FIG. 15 according to an embodiment of the invention.

FIG. 17 is a magnified cross sectional view of the catheter of FIG. 15 according to an embodiment of the invention.

### DETAILED DESCRIPTION

The invention provides a reliable system for in situ positioning of an intravenous biosensor. A catheter such as multilumen catheter, a central venous catheter (CVC), a peripherally inserted central catheter (PICC), or other commonly used peripheral intravenous (IV) line may provide a suitable platform for effective intravenous positioning of a biosensor. Although the invention may be employed using any of these types of devices, for purposes of illustration only, the invention is presented with reference to use with a multilumen CVC. One advantage of using a CVC as a platform for installing an intravenous biosensor may be its ability to reach the largest blood vessels of the body where a biosensor may be exposed to an abundant flow of blood. Further, certain embodiments of the invention may be economically employed for use with multilumen catheters. Thus, the invention is intended to have universal application to catheters.

The invention attaches, or integrates, a biosensor within a catheter. More specifically, the invention provides a system for reliably mounting a biosensor to the catheter or within a lumen of a catheter without increasing the catheter outer diameter. The invention provides for secure mounting and displacement of the biosensor from an inner wall of the catheter so that it may withstand mechanical stress during installation, and after installation receive an unimpeded flow of blood for sustained measurement accuracy.

One embodiment of the invention may employ an amperometric biosensor manufactured using flex circuit technology. Flex circuits have been used in medical devices as microelectrode substrates for in vivo applications. For example, one flex circuit design uses a laminate of a conductive foil (e.g., copper) on a flexible dielectric substrate (e.g., polyamide). The flex circuit may be formed on the conductive foil using masking and photolithography techniques. Flex circuits are desirable due to their small size, low manufacturing cost, ease in design integration, and physical flexibility during transport in applications such as CVC insertion. In one embodiment, the invention may employ a flex circuit having a length between about 1.00 inches and about 3.00 inches, and having a width between about 0.020 inches and about 0.040 inches.

A biosensor integrated with a catheter may be formed on a flex circuit substrate having electrodes mounted thereon, wherein one electrode may be an enzyme-bearing electrode. In one embodiment, the biosensor may be a glucose sensor, and the enzyme electrode may be at least partially coated with a glucose oxidase enzyme. Under proper conditions, when the enzyme electrode is energized and exposed to a flow of blood, oxygen and glucose may react with the enzyme, resulting in an output of electrical current that is proportional to the concentration of glucose in the blood. Energization of the enzyme electrode and detection of the resulting electrical signal may be achieved by connecting the electrode to external electronics via electrical wires. In addition to glucose monitoring, other biosensors may be used in the invention, such as sensors that measure electrolyte levels in blood or other analytes found in various body fluids.

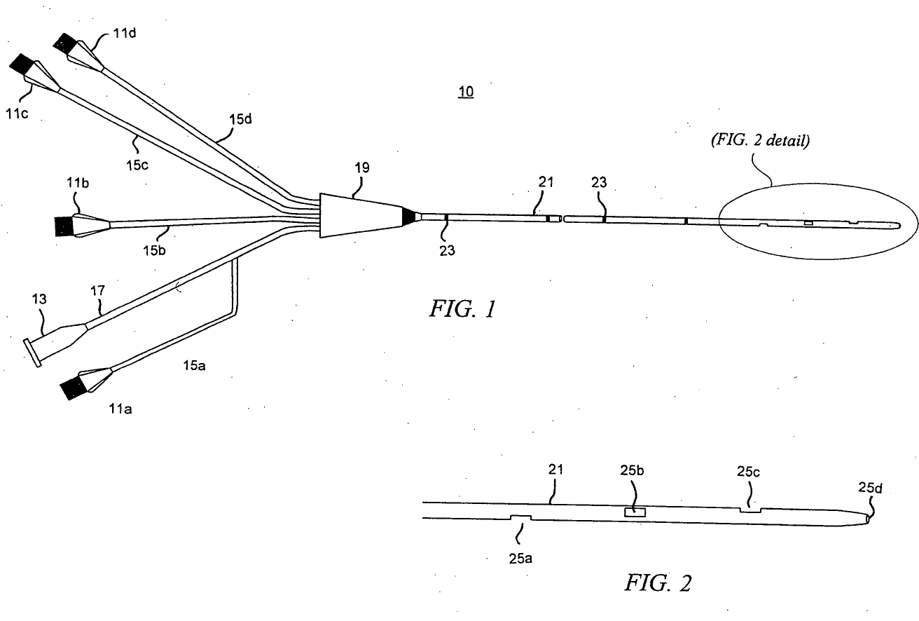

FIG. 1 shows integrating a biosensor within a multilumen catheter assembly. The catheter assembly 10 may include multiple infusion ports 11a, 11b, 11c, 11d and one or more electrical connectors 13 at its most proximal end. A lumen 15a, 15b, 15c or 15d may connect each infusion port 11a, 11b, 11c, or 11d, respectively, to a junction 19. Similarly, the conduit 17 may connect an electrical connector 13 to the junction 19, and may terminate at junction 19, or at one of the lumens 15a-15d (as shown). Although the particular embodiment shown in FIG. 1 is a multilumen catheter having four lumens and one electrical connector, other embodiments having other combinations of lumens and connectors are possible within the scope of the invention, including a single lumen catheter, a catheter having multiple electrical connectors, etc. In another embodiment, one of the lumens and the electrical connector may be reserved for a probe or other biosensor mounting device, or one of the lumens may be open at its proximal end and designated for insertion of the probe or biosensor mounting device. The details of the probe and other devices for mounting a biosensor will be further explained below.

The junction 19 connects the lumens 11a-11d and the conduit 17 to a narrow elongated tube 21 that forms an intravenous insertion portion of the catheter assembly 10. The tube 21 may be typically cylindrical, having a circular or somewhat oval cross section defining a longitudinal axis extending therethrough. The tube 21 may be formed from any material, including synthetic materials such as silicone, polyurethane, polyethylene, and the like. Through the junction 19, each of the lumens 11a-11d extend in separate parallel paths for some distance into the distal end of tube 21. One or more support structures 23 within the tube 21 may be disposed along the length of the catheter to provide rigidity.

The distal end of the catheter assembly 10 is shown in greater detail in FIG. 2. At one or more intermediate locations along the distal end, the tube 21 may define one or more ports formed through its outer wall. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d.

A port 25 exposing an active portion of a biosensor 29 may be referred to as a sensing port. A sensing port 25 may perforate an outer wall of catheter 10 to form a hole that opens into a lumen. In one embodiment, the sensing port 25 opens into only one lumen. The sensing port 25 as described herein may be generally oval or rectangular in shape, having a length between about 5.0 mm and about 15.0 mm, and having a maximum width between about 1.0 mm and about 3.0 mm. The sensing port 25 may be formed in a catheter, for example, by skiving an area of the outer wall of tube 21.

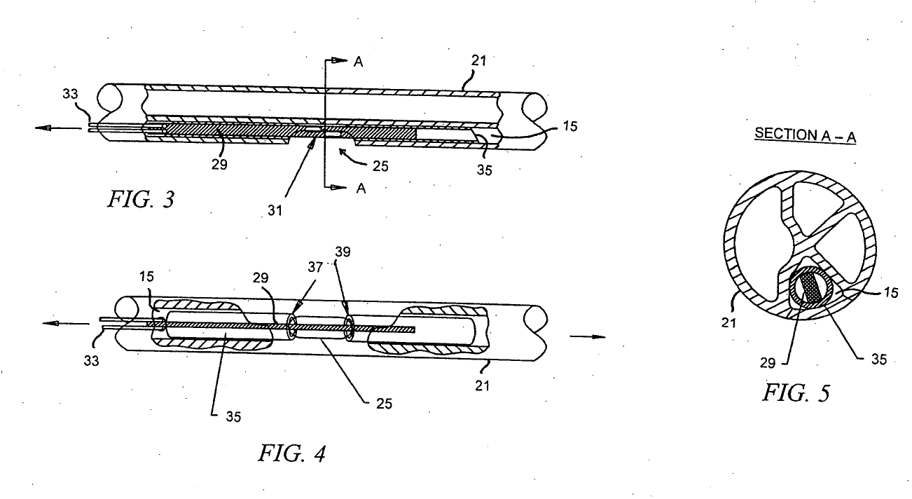

In one embodiment, one or more sensing ports 25 may be located on the tube 21 proximally to an end port. In another embodiment, a catheter may be configured with a single sensing port that is proximal to all other ports, such as port 25a of FIG. 3. In operation within a venous location, the most proximal sensing port of the catheter may lie advantageously upstream of the distal ports, so that any infusion fluids introduced into the bloodstream through a distal port are prevented from affecting biosensor measurements.

The embodiment of FIG. 3 shows a magnified transparent side view of an intermediate portion of the distal end of the tube 21 in the vicinity of the sensing port 25. In the orientation shown, a lumen 15 extends longitudinally within tube 21 along the bottom portion of the catheter. A biosensor 29 may be positioned within the lumen 15 such that its active portion 31, i.e. the portion containing an enzyme electrode, may be exposed to space outside the tube 21 through the port 25. At the proximal end of the biosensor 29, the electrical wires 33 coupled to the enzyme electrode extend from the biosensor 29 through the lumen 15. The electrical wires 33 are coupled to, or provide, a conductive path through the lumen 15 and the conduit 17 that may terminate at the electrical connector 13. In one embodiment, the electrical wires 33 may be bonded to the substrate of the biosensor 29 at a proximal location on the substrate having an area of about 0.15 square inches to about 0.30 square inches. A suitable adhesive such as Loctite 401 may be used to affect this bond.

As shown in FIG. 3, the biosensor 29 may be connected or mounted inside a length of support tubing 35. The support tubing 35 may be formed of material of a desired rigidity similar to the tube 21. The support tubing 35 may be inserted within the lumen 15 such that it spans the sensing port and positions the active portion 31 of the biosensor 29 facing radially outward and displaced from an inner wall of the catheter.

FIG. 4 is a bottom view of the intermediate portion of the tube 21 of FIG. 3. FIG. 5 shows a cross sectional view of the tube 21 corresponding to section A-A. As shown in these figures, the support tubing 35 may be positioned concentrically within the lumen 15, and the biosensor 29 may be mounted concentrically within the support tubing 35. With such an arrangement, the biosensor 29 may be effectively shielded from damage when the biosensor is positioned within the catheter, during which time frictional forces may act between the inner diameter of the lumen 15 and the outer diameter of the support tubing 35, but not on the active portion 31 of the biosensor due to its displacement from the inner diameter of the lumen 15.

After positioning the support tubing 35, to ensure that the biosensor 29 remains firmly anchored at the sensing port 25, an adhesive agent (not shown) such as an epoxy may be applied at locations 37 and 39, which correspond to the proximal and distal ends, respectively, of the sensing port 25. The adhesive may bond the biosensor 29 to support the tubing 35, and also bond support tubing 35 to the inner walls of the lumen 15. The adhesive may also beneficially seal the lumen 15 to prevent fluid or other material from entering the catheter interior through the sensing port 25. Thus, a completed catheter assembly 10 may provide an integral biosensor that is protectively centrally oriented within a lumen and exposed through a sealed sensing port in the outer catheter wall.

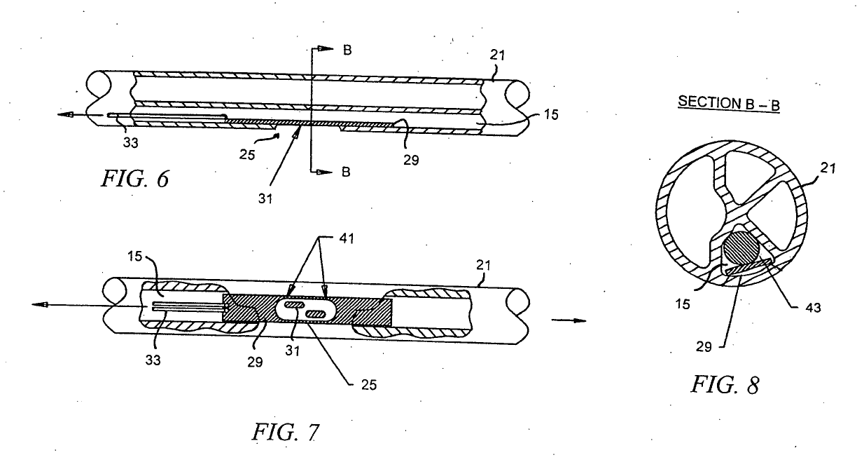

FIGS. 6, 7 and 8 illustrate another embodiment of a catheter assembly with integral biosensor according to an embodiment of the invention. These figures show alternative magnified side, bottom and cross sectional views, respectively, of the intermediate portion of the tube 21 of FIG. 3. As in a previous embodiment, a sensing port 25 may be formed at an intermediate location along a distal end of a catheter tube 21, and may be located proximally with respect to all other ports formed in the outer wall of the tube 21. In this embodiment, as shown in FIG. 6, a biosensor 29 may be mounted directly to an inner diameter of the lumen 15 at its furthest radial distance from the longitudinal axis of the tube 21 (or equivalently, to an inner diameter of the outer wall of the tube 21) such that its active portion 31 is exposed through the sensing port 25 and displaced radially inwardly from the outer diameter of the tube 21. In other words, in this configuration the active portion 31 of biosensor 29 may form an outer diameter of the catheter at the location of the sensing port 25 that is inwardly displaced a small distance less than the outer diameter of adjacent areas of the outer wall of the tube 21.

Prior to positioning of the biosensor 29, it may be mounted to a support member 43, which may be a tube or rod having a cylindrical or trapezoidal cross section. The support member 43 may then be inserted through the lumen 15 until the active portion 31 of the biosensor 29 is properly exposed through the sensing port 25. As shown in the cross sectional view of FIG. 8, the support member 43 may abut an inner radial wall of the lumen 15 and place the biosensor 29 in a position facing the opposite outer wall.

One advantage to embodiment of FIG. 6 is that it allows for simplified sealing of the sensing port. By mounting the biosensor 29 flush against the inner wall of the lumen 15, a circumferential interface 41 is created at the border of the sensing port 25 and the outwardly facing surface of the biosensor 29. The interface 41 may be sealed with a single bead of an appropriate sealant or bonding agent to prevent fluid and foreign materials from entering the lumen 15 through the sensing port 25. Another advantage of this embodiment is that placement of the biosensor directly adjacent to the outer diameter of the catheter may provide better exposure to blood flow.

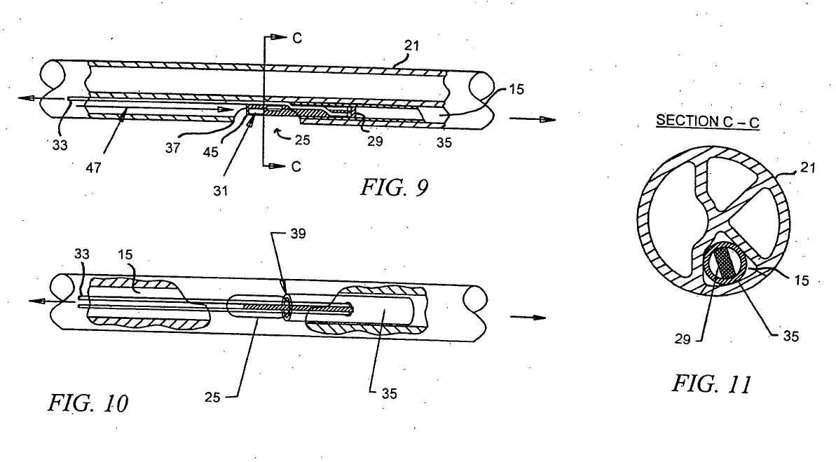

FIGS. 9, 10 and 11 illustrate an embodiment of a catheter assembly according to an embodiment of the invention which allows an integral biosensor to be flushed with an IV solution, whether the catheter is withdrawn or in situ. These figures show alternative magnified side, bottom and cross sectional views, respectively, of the intermediate portion of tube 21 of FIG. 3. As in previous embodiments, a sensing port 25 may be formed at an intermediate location along a distal end of a catheter tube 21, and may lie most proximally with respect to any other infusion port formed in an outer wall of the tube 21. As in the embodiment of FIG. 3, a support tubing 35 may be included to mount and position a biosensor 29 so that its active portion 31 is exposed through the sensing port 25 and displaced from the inner diameter of the lumen 15. In this embodiment, the support tubing 35 may be positioned such that the proximal end 45 of the biosensor 29 is located distally with respect to the proximal end 37 of the sensing port 25. This configuration allows for a flow 47 of an IV solution (such as saline or other cleansing solution) to be injected into the lumen 15 (e.g. through an infusion port 11a) and ejected from the catheter through the sensing port 25. In this manner, the cleansing fluid may advantageously flush the active portion 31 of the biosensor 29 and thereby remove clotted blood or other materials from the surface of the biosensor that may adversely affect its operation. A sealant may be applied at the distal end 39 of the sensing port 25 to bond the biosensor 29 to support the tubing 35, and to seal the distal portion of the lumen 15.

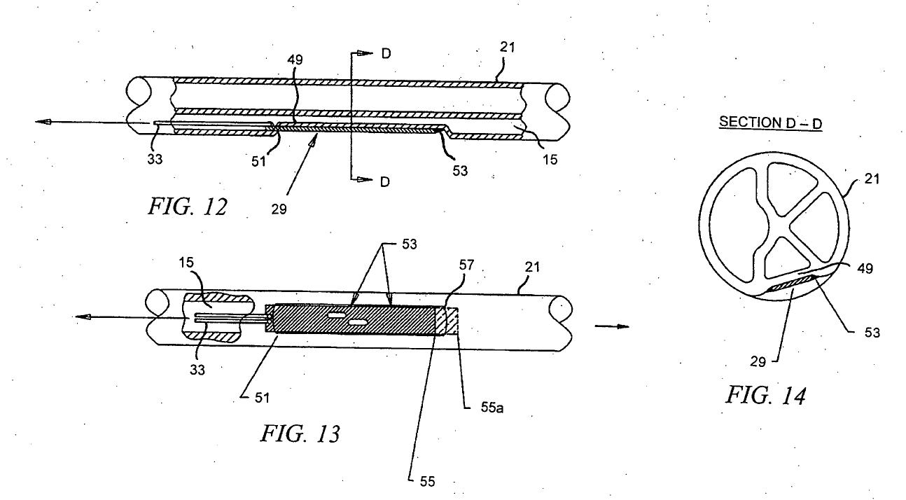

FIGS. 12-14 illustrate another embodiment of a catheter with integral biosensor according to an embodiment of the invention. These figures show an alternative set of magnified side, bottom and cross sectional views, respectively, of the intermediate portion of the tube 21 of FIG. 3. Using this arrangement, the biosensor 29 may be exposed to a flow of blood by mounting it directly to an outer wall of the catheter without having to form a sensing port through the tube 21.

To biosensor may not increase the overall outer diameter of the catheter because the biosensor 29 is mounted in a recessed area of the tube 21. The side view of FIG. 12 shows one example of a generally rectangular recessed area 49 formed on the outer wall of the catheter between proximal and distal ends of the tube 21. The recessed area 49 may be located proximally with respect to one or more intermediate ports formed in the outer wall of the tube 21, and may be the most proximal of all such ports. A lumen 15 may extend longitudinally through tube 41 and form an inner wall bordering the recessed area. In one embodiment, the recessed area 49 may be formed in a manufactured catheter by heating and pressing a portion of the tube 21. In another embodiment, the recessed area 49 may be formed during catheter fabrication by molding.

A mounting port 51 may be formed through a proximal, substantially transverse wall of the recessed area 49, as indicated. A biosensor 29, such as a thin flex circuit amperometric biosensor, may extend through the mounting port 51 along the surface of the recessed area 49, such that a portion of the proximal end 37 of the biosensor 29 remains inside the lumen 15. The portion of the proximal end 37 remaining within the lumen 15 may include at least an area sufficient for coupling the wires 33 to the biosensor 29. The distal end 55 of the biosensor 29 may abut a substantially transverse distal wall of the recessed area 49. An adhesive or sealant 53 may then complete the assembly. The sealant 53 may be applied to the area in and around the mounting port 51 to provide a seal preventing passage of fluid therethrough. The sealant 53 may also be applied to the edges and bottom surface of the biosensor 29 to securely bond it to the recessed area 49.

In an alternative embodiment indicated in FIG. 13, a second mounting port 57 may be formed in the transverse distal wall of the recessed area 49. In this option, the distal end of the biosensor 29, indicated by dashed portion 55a, extends into the lumen 15 through the second mounting port 57. The sealant 53 may then be applied to the second mounting port area to seal the lumen 15 at the location of the mounting port 57. This arrangement may provide a stronger and more reliable means for fastening the biosensor to the catheter.

As shown in FIG. 14, the mounting arrangement for either option (i.e. one or two mounting ports) allows the biosensor to be installed on the outer wall of the catheter without increasing the area of the catheter cross section. This installation further protects the biosensor from frictional forces by placing the outermost surface of the biosensor at a radial distance from the axis of the tube 21 that is less than the radius of the tube's outer diameter.

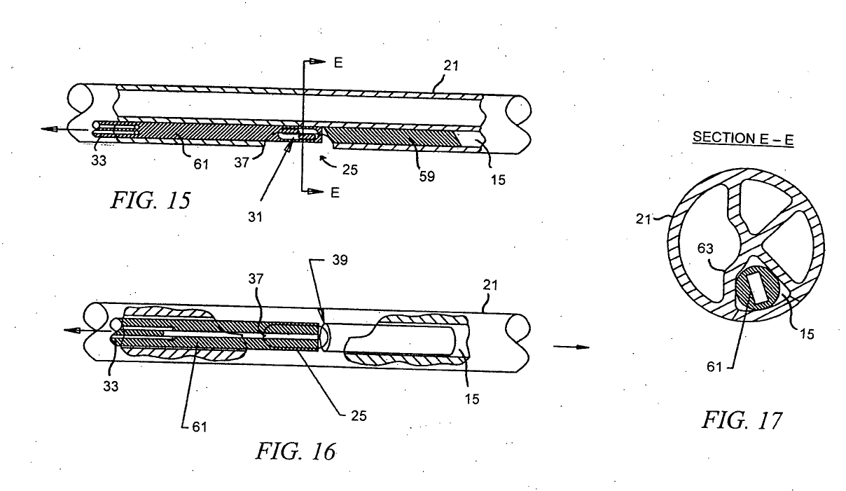

Another embodiment of a catheter with integral biosensor is depicted in FIGS. 15-17. As in previous embodiments, a sensing port 25 may be formed at an intermediate location along a distal end of a catheter tube 21, which location may be proximal to one or more fluid ejection ports. In this embodiment, a biosensor having an active portion 31 is integrated with a probe 61. The probe 61 may be a rod or tubing formed from a flexible substance such as vinyl, urethane, nylon or other suitable material. In one embodiment, the probe 61 may be formed from a material that may be bonded to a flex circuit substrate. The wires 33 for energizing and sensing of the integral biosensor may extend from the proximal end of the probe 61 and terminated at a connector 13.

The flexibility of probe 61 allows it to be inserted into a lumen 15 at a proximal location, such as through an infusion port 11a, and moved through lumen until it reaches a sensing port 25. A plug 59 may be inserted in the distal end of lumen 15, as shown, to stop the progress of the probe 61 so that the active portion 31 may be accurately positioned at the sensing port 25. A keying configuration 63 may be formed in the inner wall of the lumen 15 to ensure proper orientation of the probe 61 within the lumen 15 so that the active portion 31 faces outward through the sensing port 25 for optimal exposure to blood flow. Thus, during installation, the key 63 guides the probe through the lumen 15 in proper orientation to exposes the active portion 31 through the sensing port 25 when a distal end of the probe 61 reaches the plug 59.

As indicated in FIGS. 15-17, the active portion 31 may be protected from frictional forces by mounting it concentrically with respect to the probe 61 so that during installation, only the outer diameter of the probe 61 comes into contact with the inner wall of the lumen 15. After inserting the probe 61, the assembly may be completed by sealing the proximal end 37 and distal end 39 of the sensing port 25 with an appropriate sealant. In one embodiment, where the probe 61 forms a tight compression fit against the inner wall of the lumen 15, a sealant may not be required at one or both ends 37 and 39.

The invention has been disclosed in an illustrative manner. Accordingly, the terminology employed throughout should be read in an exemplary rather than a limiting manner. Although minor modifications of the invention will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A catheter for detecting a physiological parameter in a blood vessel, comprising:
an elongated tube having a longitudinal axis;
a sensing port perforating an outer wall of the tube between proximal and distal ends of the tube;
at least one lumen extending longitudinally through the tube and connecting to the sensing port, the lumen having a longitudinal axis offset from the longitudinal axis of the tube;
a support member spanning the sensing port and positioned concentrically within the lumen; and
a biosensor connected to the support member and exposed through the sensing port.

2. The catheter of claim 1, wherein the support member displaces an active portion of the biosensor from an inner wall of the catheter.

3. The catheter of claim 1, wherein the biosensor is mounted concentrically within the support member.

4. The catheter of claim 1, wherein the sensor is mounted to an inner diameter of the outer wall of the tube.

5. The catheter of claim 1, further comprising sealant to prevent passage of fluid into the lumen through the sensing port.

6. The catheter of claim 1, wherein the lumen is sealed at a distal end of the sensing port to prevent passage of fluid through the sensing port into the distal end of the lumen, and wherein the lumen opens to a proximal end of the sensing port to allow passage of fluid from the lumen through the proximal end of the sensing port.

7. The catheter of claim 1, further comprising one or more intermediate ports formed in the outer wall of the tube distally with respect to the sensing port.

8. The catheter of claim 7, wherein the sensing port is proximal to all other ports formed in the outer wall of the tube.

9. A catheter for detecting a physiological parameter in a blood vessel, comprising:
an elongated tube;
a recessed area formed on an outer wall of the tube between proximal and distal ends of the tube;
at least one lumen extending longitudinally through the tube and forming an inner wall of the recessed area;
a mounting port formed through a transverse proximal wall of the recessed area; and
a biosensor extending through the mounting port and bonded to the outer wall of the tube on the recessed area.

10. The catheter of claim 9, wherein the mounting port is sealed to prevent passage of fluid therethrough.

11. The catheter of claim 9, further comprising a second mounting port formed through a transverse distal wall of the recessed area, the biosensor extending through the second mounting port.

12. The catheter of claim 11, wherein the first and second mounting ports are sealed to prevent passage of fluid therethrough.

13. The catheter of claim 9, further comprising one or more intermediate ports formed in the outer wall of the tube distally with respect to the recessed area.

14. The catheter of claim 13, wherein the recessed area is proximal to all other ports formed in the outer wall of the tube.

15. The catheter of claim 9, wherein an outermost surface of the biosensor is displaced a radial distance from the axis of the tube that is less than the radius of the outer wall of the tube.

16. A catheter for detecting a physiological parameter in a blood vessel, comprising:
an elongated tube having a longitudinal axis;
a sensing port perforating an outer wall of the tube between proximal and distal ends of the tube;
at least one lumen extending from a proximal end of the tube longitudinally through the tube and terminating at a distal end of the sensing port, the lumen having a longitudinal axis offset from the longitudinal axis of the tube;
a probe extending through the lumen to the sensing port; and
a biosensor connected to the probe and exposed through the sensing port.

17. The catheter of claim 16, wherein the probe displaces an active portion of the biosensor from an inner wall of the catheter.

18. The catheter of claim 16, wherein the biosensor is mounted concentrically with respect to the probe.

19. The catheter of claim 16, further comprising a plug positioned in the lumen at a distal end of the sensing port.

20. The catheter of claim 19, wherein the lumen is keyed to guide the probe through the lumen in proper orientation to exposes an active portion of the biosensor through the sensing port when a distal end of the probe reaches the plug.
